(19) United States
(12) Patent Application Publication
(10) Pub. No.: US 2026/0000607 A1
(43) Pub. Date: Jan. 1, 2026

(54) Polymer-polymer Matrix for the Stabilization of Exosomes

(71) Applicants: Babak Ghalili, New York, NY (US); Keyon Janani, Baton Rouge, LA (US); Michael J. Borja, Keyport, NJ (US)

(72) Inventors: Babak Ghalili, New York, NY (US); Keyon Janani, Baton Rouge, LA (US); Michael J. Borja, Keyport, NJ (US)

(21) Appl. No.: 19/242,279

(22) Filed: Jun. 18, 2025

Related U.S. Application Data
(60) Provisional application No. 63/664,764, filed on Jun. 27, 2024.

(51) Int. Cl. A61K 9/06; A61K 9/70; A61K 35/28; A61K 47/10; A61K 47/18; A61K 47/36

(52) U.S. Cl. CPC A61K 9/06; A61K 9/7007; A61K 35/28; A61K 47/10; A61K 47/183; A61K 47/186; A61K 47/36

(57) ABSTRACT

The present disclosure relates to exosomes and compositions including one or more natural or synthetic sulfated polymers crosslinked with one or more crosslinking agent cationic polyamines as well as methods of use and methods of making of them.

### FIELD

The aspects of the present disclosure relate to an exosome composition and methods of making and methods of using it.

### BACKGROUND

Stem-Cell exosomes are extra-cellular vesicles that are secreted by mesenchymal stem cells into the cell culture medium. They typically range in size from 30 nm-150 nm. The exosomes themselves contain a variety of microRNAs (miRNA), cytokines, and growth factors. Exosomes have the ability to fuse with other cells of the human body and release their content directly into the merging cell. Therefore, no uptake mechanisms for these exosomes are necessary in order for them to fuse with a target cell.

Together with the exosomes, stem cells also secrete cytokines, growth factors and a multitude of other proteins and peptides directly into the surrounding culture medium. In general, the entirety of all molecules released by stem cells can be summarized as “secretome”. Depending on the method of culture medium collection and refinement, the composition of the final product can be comprised of pure exosomes or a preparation that contains exosomes, and stem cell secreted molecules like cytokines, growth factors and proteins. The exact composition of the “secretome” varies and depends on the stem cell type/origin, culture conditions, and methods of concentration and buffer exchange.

One of the challenges for stabilizing exosomes is to keep them from agglomeration. Once agglomerated, exosomes lose efficacy if the agglomerated particle size exceeds 200 nm (nanometers). Most practices cryogenically freeze the exosome liquid to prevent them from sticking together. Before using, the exosomes need to be thawed and used immediately, either topically or injected or else they must be discarded.

Studies have shown that exosomes derived from mesenchymal stem cells (MSCs) can modulate the inflammatory response of wounds by promoting the polarization of macrophages and enhancing the secretion of anti-inflammatory factors, thereby facilitating wound healing. Also, MSCs-derived exosomes can promote bone regeneration by directly transferring their growth factors and subsequently controlling downstream signaling pathways in the targeted cells. Exosomes also can regulate immune responses, inhibit osteoclast activities and induce osteogenesis and angiogenesis.

### SUMMARY

In one embodiment, a composition is provided. The composition includes a plurality of exosomes; and a polymer-polymer complex including one or more natural or synthetic sulfated polymers crosslinked with one or more crosslinking agent cationic polyamines.

In another embodiment, a method is provided. The method includes using a composition including a plurality of exosomes; and a polymer-polymer complex including one or more natural or synthetic sulfated polymers crosslinked with one or more crosslinking agent cationic polyamines and applying the composition to a cut, scrape, wound, abrasion, burn or other external wound of an animal

In another embodiment, a method of making an exosome composition that maintains the structure and integrity of the exosomes is provided. The method includes mixing a plurality of exosomes, a polymer-polymer complex including one or more natural or synthetic sulfated polymers crosslinked with one or more crosslinking agent cationic polyamines to the first mixture to form a second mixture and a lipid that forms liposomes without the use of alcohol to the second mixture.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by embodiments of the present disclosure. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

The terms “%”, “% by weight”, “weight %” and “wt %” are all intended to mean unless otherwise stated, percents by weight based upon a total weight of 100% end composition weight. Thus 10% by weight means that the component constitutes 10 wt. parts out of every 100 wt. parts of total composition.

The term “pharmaceutically acceptable” means that which is useful in preparing a pharmaceutical composition that is generally non-toxic and is not biologically undesirable and includes that which is acceptable for veterinary use and/or human pharmaceutical use.

The term “topically acceptable” means the compound, substance or device may be administered to or onto the surface of a patient, including the skin or other accessible tissues, without substantial harmful effects to the body part and/or its surfaces.

The terms “treating” and “effective amount”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

All of the embodiments included here are with the proviso that the sum of ingredients in the exemplary compositions does not exceed 100%.

The aspects of the present disclosure relate to exosome mixtures, systems and compositions and preservative systems and compositions and methods of forming them in which the viable (i.e., active) structure and integrity of the exosomes and its contents can be maintained using additional components to preserve the exosomes to deter disadvantageous occurrences such as, for example, aggregating or clumping and making the exosome composition more stable. Aspects of the present disclosure also include fluid mixtures and compositions of exosomes and exosome mixtures (e.g., a plurality of exosomes) including polymer-polymer complexes in combination with the exosomes to preserve and maintain the structure and integrity of the exosomes and its contents and the liposomes which include exosome mixtures and polymer-polymer complexes in combination with the exosomes internal to the liposomes and internal and external to the liposomes as well to prevent or minimize deleterious conditions such as, for example, exosomes from agglomerating aggregating or clumping and making the exosome composition more stable. Aspects of the present disclosure also include fluid mixtures and compositions of exosomes and exosome mixtures including polymer-polymer complexes including one or more natural or synthetic sulfated polymers that are crosslinked with cationic polyamines in combination with the exosomes to preserve and maintain the structure and integrity of the exosomes and its contents and the liposomes which include exosome mixtures and polymer-polymer complexes in combination with the exosomes internal to the liposomes and internal and external to the liposomes as well to prevent or minimize deleterious conditions such as, for example, exosomes from agglomerating clumping. Factors such as concentration, temperature, high shear mixing, and sonication will influence the formation of polymer-polymer complexes.

A “liposome” is a generic term encompassing a variety of single and multilamellar lipid vehicles formed by generating enclosed lipid bilayers or aggregates. Liposomes may have vesicular structures with a bilayer membrane, generally comprising a phospholipid, and an inner medium that generally comprises an aqueous composition. Liposomes for the present disclosure may include unilamellar liposomes, multilamellar liposomes, and multivesicular liposomes and may be positively charged, negatively charged, or neutrally charged.

The exosomes included in the exosome mixture or serum embodiments of the present disclosure include a plurality of exosomes and can be obtained or extracted from different sources including various cell types, for example, placental cells and stem cells. An exosome mixture can include an aqueous mixture with exosomes and an exosome serum includes a water or oil-based mixture of exosomes including, for example, one or more of water, propylene glycol, butylene glycol, glycerin, lecithin, polyvinyl pyrrolidone, mineral oil, MCT oil (medium chain triglycerides), squalane, glyceryl monostearate, phenoxyethanol, propyl paraben and methyl paraben and that has a viscosity greater than that of an exosome mixture with a viscosity ranging from about 10 mPa-s to about 100 mPa-s, about 50 mPa-s.

The exosomes from stem cells contain biomolecules that are different from the exosomes of other cells. Exosomes from urine, blood or plasma serum, or epithelial cells or differentiated cells, will not have the same regenerative effect as compared to stem cell exosomes (e.g., mesenchymal stem cells (MSCs) exosomes). Exosomes are generated by all living cells. The contents of stem cell exosomes (e.g., MSC exosomes) are dependent on where they are coming from. Stem cells have a certain regenerative effect when injected. These effects can be replicated by using exosomes from stem cells. The regenerative effect of stem cells likely comes from the exosome itself and its contents. Mesenchymal stem cell exosomes transfer functional cargos like miRNA and mRNA molecules, peptides, proteins, cytokines and lipids from MSCs to the recipient cells. These exosomes participate in intercellular communication events and contribute to the healing of injured or diseased tissues and organs. and, as a result, can be more beneficial. Exosome extraction can be performed using different known types of centrifugation and ultracentrifugation. After the centrifugation and ultracentrifugation steps, they can be further purified using additional known procedures including, for example, gel filtration. Exosomes can be present in embodiments of the exosome mixture or serum of the present disclosure in an amount of about 0.25 wt % to about 1.00 wt %, about 0.50 wt % or about 10 million to about 20 trillion exosomes, or about 1 billion exosomes per 1 ml volume in the exosome mixture.

While all mammalian cells secrete exosomes, only exosomes derived from stem cells may be currently considered therapeutic. Stem cells can be cultured in many different ways. This includes 2D culturing, which typically occurs in flasks or cylinders. Three-dimensional culturing is facilitated in bioreactors, which can include stirred tank and hollow fiber setups. Independent of the culturing method in which they are propagated of the stem cell culture, the exosomes are secreted into the culture medium by the stem cells. The culturing method can determine the concentration of exosomes per volume culture medium.

The exosomes included in embodiments of the present disclosure preferably can also be plant exosomes (also referred to as plant exosome nanoparticles, plant-derived exosome-like nanovesicles (PDENs) and plant-derived exosome-like nanovesicles (PELNs)) obtained or extracted from plant cells. Plant exosomes share similarities with mammalian-derived exosomes in terms of their structure and function. There are believed to be three possible pathways for the biogenesis of plant extracellular vesicles. They can be found in the exocyst-positive organelle pathway, the multivesicular bodies pathway, and the vacuolar pathway. Among these, the multivesicular bodies pathway is considered the main pathway for the formation of plant exosomes.

Plant-derived exosome-like nanoparticles can include various bioactive biomolecules. As an alternative cell-free therapeutic approach, they have the potential to deliver nano-bioactive compounds to the human body, and thus can lead to various anti-inflammatory, antioxidant, and anti-tumor benefits.

Exosomes from plants are membrane vesicles with nanoparticles (30-150 nm) that contain a number of bioactive biomolecules. In many applications, such as skin care, drug delivery and biomedicine, they have been shown to have multiple uses. Exosomes from plants can possess antioxidant, anti-inflammatory and anti-aging. Plant-derived natural chemicals, including plant exosomes, can be applied in cosmetics because they are beneficial to human skin, such as, for example, anti-aging, moisturizing, lightening, rejuvenating, nourishing.

Interest in the health effects of plants has recently increased due to their safety and applicability in the formulation of pharmaceuticals and cosmetics. Long-known plant materials as well as newly discovered ones are increasingly being used in natural products of plant origin. The beneficial effects of plants and plant constituents on the skin and on the human body, can include, for example, moisturizing (e.g., Cannabis sativa, Hydrangea serrata, Pradosia mutisii and Carthamus tinctorius), anti-aging (e.g., Aegopodium podagraria, Euphorbia characias, Premna odorata and Warburgia salutaris), antimicrobial (e.g., Betula pendula and Epilobium angustifolium), antioxidant (e.g., Kadsura coccinea, Rosmarinus officinalis, Rubus idaeus and Spatholobus suberectus), anti-inflammatory (e.g., Antidesma thwaitesianum, Helianthus annuus, Oenanthe javanica, Penthorum chinense, Ranunculus bulumei and Zanthoxylum bungeanum), regenerative (e.g., Aloe vera, Angelica polymorpha, Digitaria ciliaris, Glycyrrihza glabra and Marantodes pumilum), wound healing (e.g., Agrimonia eupatoria, Astragalus floccosus, Bursera morelensis, Jatropha neopauciflora and Sapindus mukorossi), photoprotective (e.g., Astragalus gombiformis, Calea fruticose, Euphorbia characias and Posoqueria latifolia) and anti-tyrosinase activity (e.g., Aerva lanata, Bruguiera gymnorhiza, Dodonaea viscosa, Lonicera japonica and Schisandra chinensis), as well as their role as excipients in cosmetics (coloring (e.g., Beta vulgaris, Centaurea cyanus, Hibiscus sabdariffa and Rubia tinctiorum), protective and aromatic agents (e.g., Hyssopus officinalis, Melaleuca alternifolia, Pelargonium graveolens and Verbena officinalis).

Animal or plant exosomes can also be used to transport drugs directly to the target cells, potentially transforming drug delivery methods. On the whole, exosomes from animal and plant exosomes can potentially be exploited in many different applications in medicine, biotechnology and cosmetics, and more.

In order to isolate stem cell derived exosomes, the conditioned culture medium undergoes a procedure that concentrates the exosomes and ultimately removes the culture medium which then can be replaced with the desired medium for various pharmaceutical and/or cosmetic compositions and uses, e.g., 0.9 wt % sodium chloride or phosphate buffered saline for other applications such as cosmetics. The methods for exosome isolation include but are not limited to the following:

### 1. Ultracentrifugation

a. Conditioned medium is centrifuged at lower speeds to remove cell debris.

b. Then, the remaining supernatant is centrifuged at high g forces (e.g., 100,000×g) to facilitate the sedimentation of the exosomes.

c. Exosome pellets are then washed and resuspended in the desired medium.

### 2. Sucrose Density Centrifugation

a. Exosomes are separated in a density gradient established by different sucrose concentrations.

b. The exosome fraction is then isolated and further processed by washing and resuspension in the desired medium.

### 3. Size Exclusion Chromatography

a. Conditioned medium is loaded onto a column that allows for the separation of exosomes from other particles based on their size.

### 4. Precipitation

a. Conditioned medium is exposed to agents that lower the solubility of exosomes and thereby facilitate their precipitation.

b. This agent can be polyethylene glycol (PEG) or other suitable polymers or compounds.

c. Exosomes are then washed and can be resuspended in the medium of choice.

### 5. Tangential Flow Filtration/Ultrafiltration

a. Conditioned medium is filtered using tangential flow filters.

b. Exosomes are retained in the filter (retentate)

c. The exosomes are washed while conditioned medium is removed.

d. Exosomes are suspended in the medium of choice.

### 6. Immunoprecipitation

a. Exosomes are precipitated from conditioned medium using antibodies targeted to specific exosome proteins.

b. Exosomes are then released, washed, and resuspended in desired medium.

7. Immunoaffinity/with or without chromatography

a. Exosomes are retained on a column or other physical support by antibodies that are targeted to unique exosome proteins.

b. Exosomes are then washed, released from support, and resuspended in desired medium.

8. Flow-field flow fractionation

a. Originated as a detection method for exosome populations, it has been recently used to isolate exosomes from conditioned medium.

Liposomes included in the present disclosure are lipid vessels that can contain stabilizing agents, preservatives, and penetration enhancers. Such liposomes have at least one lipid bilayer and can be formed, for example, using lecithin without the use of ethanol or other alcohol because of the latter's adverse effects on exosomes. Other than lecithin (phospholipid aka. Phosphatidylcholine), other examples of lipids that can be used to form liposomes used in embodiments of the present disclosure are phosphatidic acid (phosphatidate) (PA), Phosphatidylethanolamine (cephalin) (PE), Phosphatidylserine (PS), Phosphatidylinositol (PI), Phosphatidylinositol phosphate (PIP), Phosphatidylinositol bisphosphate (PIP2) and Phosphatidylinositol trisphosphate (PIP3), Phosphosphingolipids, Ceramide phosphorylcholine (Sphingomyelin) (SPH), Ceramide phosphorylethanolamine (Sphingomyelin) (Cer-PE), Ceramide phosphoryllipid etc. Additionally, niosomes can also be used in place or in combination with liposomes even though niosomes may not as stable as liposomes. Niosomes are vesicles composed of hydrated non-ionic surfactants, such as alkyl-ether, esters, and amides, and cholesterol and can be used as an alternative to liposomes. Niosomes can be made using the same process as is used in making liposomes.

The polymer-polymer complex of the present disclosure includes one or more natural or synthetic sulfated polymers crosslinked with one or more crosslinking agent cationic polyamines.

One part of the polymer-polymer complexes can include natural and synthetic sulfated polymers. The natural and sulfated polymers can include, for example, carrageennan, fucoidan, ulvan, alginates, galactans, laminarians, sulfated carboxymethyl cellulose, sulfated polyvinyl alcohol (PVAS), sulfated copolymers of acrylic acid with vinyl alcohol (PAVAS) and mixtures thereof. Examples of natural sulfated polymers include E-poly-L-lysine, putrescine (PUT), spermine (SPE), and spermidine (SPD), polyureas, coco-amine acetate, N-dodecyl-1,3-propanediamine and bis (3-aminopropyl) dodecylamine, and urea. Examples of synthetic sulfated polymers include ethylenediamine, macrocyclic polyamines, tris (2-aminoethyl) amine, polyethylenimine, 1,3,5-triazinane and mixtures thereof. The synthetic sulfated polymers can include, for example, poly (styrenesulfonic acid) (pSS), poly (vinylsulfonic acid) (pVS), sodium dextran sulfate (DS), sodium polystyrene sulfonate (PSS), sodium polyanethole sulfonate (PAS), and potassium polyvinyl sulfate (PVS) and mixtures thereof.

The second part of the polymer-polymer complexes can include crosslinking agent cationic polyamines including, for example, ε-poly-L-lysine, putrescine (PUT), spermine (SPE), and spermidine (SPD), polyureas, coco-amine acetate, N-dodecyl-1,3-propanediamine and bis (3-aminopropyl) dodecylamine, urea and mixtures thereof.

One embodiment of the present disclosure includes a ratio of (a) about 1 molar part natural or sulfated polymers, e.g. carrageenan, to (b) 0.05 molar part to about 1.00 molar part crosslinking agent polyamine compound.

Carrageenan is a sulfated galactan that form helical structures. It is composed of d-galactose units, alternating 3-linked β-D-galactopyranose (G-units) and 4-linked α-D-galactopyranose (D-units) or 4-linked 3,6-anhydro-α-D-galactopyranose (DA-units), forming the disaccharide repeating unit of carrageenans. There are three main commercial classes of carrageenan. Kappa forms strong, rigid gels in the presence of potassium ions, and reacts with dairy proteins. It is sourced mainly from Kappaphycus alvarezii. lota forms soft gels in the presence of calcium ions. It is produced mainly from Eucheuma denticulatum. Lambda does not gel, and is used to thicken dairy products. These can give carrageenans the ability to form a variety of different gels at room temperature. They are widely used in food and other industries as thickening and stabilizing agents.

The resulting mixture including exosome, optionally including liposome and/or niosomes (inside the liposome and/or niosomes, outside the liposome and/or niosomes or both) and additional components (e.g., a solvent/skin penetrant (e.g., DMSO) and preservative (e.g., epsilon poly L-lysine)) can be maintained for storage prior to use at a temperature of from about −200° C. to about room temperature (about 25° C.). The exosome, a solvent/skin penetrant (e.g., DMSO) and preservative (e.g., epsilon poly L-lysine) composition, optionally including liposomes, embodiments of the present disclosure could maintain the structure and integrity of the exosomes for a time period for as much as two years at a temperature of at most about 4.4° C.

DMSO can also be included in embodiments of the present disclosure and is a polar aprotic solvent that is preferably included in embodiments of the present disclosure and can deter the presence of clumping of the exosomes as well as act as a solvent/skin penetrant in embodiments of the present disclosure. DMSO can be present in embodiments of the present disclosure in an amount of about 0.001 wt % to about 0.50 wt %, but no more. Other examples of a solvent/skin penetrant that can be used in embodiments of the present disclosure are acetone, DMF (N,N-dimethylformamide), acetonitrile, HMF (hydroxymethylfurfural), crown ethers, fatty acids, essential oils, urea, azone, sodium PCA, etc. These other solvents/skin penetrants can be present in embodiments of the present disclosure in an amount of about 0.001 wt % to about 0.50 wt %, but no more.

Linatural can also be included in embodiments of the present disclosure and is a combination of propylene glycol, potassium sorbate, and ethylhexyl glycerin and has been widely used in the cosmetic industry. Linatural can be present in embodiments of the present disclosure in an amount of about 0.5 wt % to about 2.0 wt %.

Embodiments of the present disclosure can also include one or more preservatives. For example, phenoxyethanol is a preservative used in many cosmetics and personal care products. Phenoxyethanol can be present in embodiments of the present disclosure in an amount of about 0.001 wt % to about 1.00 wt %. (1.0 wt % is the maximum currently allowed by the FDA for phenoxyethanol.)

Other examples of a preservative that can be used in embodiments of the present disclosure are K sorbate, aminobenzoate esters, quaternary ammonium cations and/or compounds, (BZK), benzoic acid/salts, benzyl alcohol, chlorhexidine, chlorocresol, imidurea, bronopol, propionic acid/salts, sorbic acid/salts, phenol, acetate, borates, nitrates etc. The amount of these preservatives should be no more than 0.015 wt %.

Embodiments of the present disclosure may also include one or more humectant/emollients, such for example, butylene glycol, shea butter, squalane, and fatty alcohols like cetyl alcohol hyaluronic acid, glycerin, aloe, elastin, and collagen, glycerin, propylene glycol etc. The one or more humectant/emollients can be present in embodiments of the present disclosure in an amount of about 0.10 wt % to about 10.0 wt %, about 2.0 wt % to about 6.0 wt %, about 4.0 wt %.

Embodiments of the present disclosure may also include one or more excipients, such for example, hemp oil (e,g., full spectrum hemp oil), aloe, vitamins, natural fruit extracts, panthenol, tocopherol acetate, ascorbic acid, niacinamide, menthol, biotin etc. One or more excipients can be present in embodiments of the present disclosure in an amount of about 0.01 wt % to about 10.0 wt %, about 2.5 wt % to about 7.5 wt %, about 5.0 wt %. Other excipients can include thickeners including, for example, natural/synthetic gums i.e., xanthan, algin, CMC, PVP, PVOH, HPMC, methyl cellulose, gum Arabic/acacia, karaya, MVE-MA copolymer, carbomer, etc. The other excipients can be present in embodiments of the present disclosure in an amount of about 0.01 wt % to about 3.0 wt %, about 0.10 wt % to about 1.0 wt %, about 0.2 wt %.

One or more chelating agents can also be optionally added such as for example, citric acid, EDTA, dimercaprol, succimer, penicillamine, trientine and deferrioxamine. The one or more chelating agents can be present in embodiments of the present disclosure in an amount of about 0.05% wt% to about 0.10 wt %, about 0.075 wt %.

Cannabinoids can also be included in embodiments of the present disclosure and are an active agent and a class of chemical compounds that can be derived from plants (phytocannabinoids) or synthetically produced. Cannabinoids can have local and systemic analgesic, pain relieving, pain treating and anti-inflammatory therapeutic properties. Cannabinoids may also have other medical benefits and/or be useful in treating other medical conditions including, for example, reduction of anxiety and depression, reduction of symptoms like nausea, vomiting and pain related to cancer treatments, reduction of acne, protection of the neural system and benefits for the heart and circulatory system by the lowering of blood pressure. Cannabinoids can also have therapeutic value as a nutrient and can be included in composition and method embodiments of the present disclosure in an effective amount to perform that function.

Examples of phytocannabinoids include Cannabidiol (CBD) including, for example, CBD oil, Cannabinol (CBN) and tetrahydrocannabinol (THC), the latter being a known psychotropic compound and the first two being non-psychotropic. Cannabis plants can exhibit wide variation in the quantity and type of cannabinoids they produce. Selective breeding of the plants can be used to control the genetics of plants and modify the cannabinoids produced by the plant. For example, there are strains that are used as fiber (commonly called hemp) and, as a result, have been bred such that they are low in psychoactive chemicals like THC. Such strains (e.g., hemp) used in medicine are, for example, often bred for high CBD content and cannabinoids included herein (unless otherwise stated) have minimal levels of THC (less than 0.3 wt %). Examples of oral or pharmaceutically effective cannabinoids include CBD (for example, full spectrum hemp or CBD oil). Cannabinoid, including, for example, phytocannabinoids including CBD or full spectrum hemp or CBD oil, can be in an amount of about 0.1 wt % to about 20 wt %, about 0.1 wt % to about 10 wt %, about 0.5 wt % to about 6 wt % or about 5.7 wt %. CBD can be in an amount of about 0.1 wt % to about 20 wt %, about 0.1 wt % to about 10 wt %, about 0.5 wt % to about 5 wt %, about 0.5 wt % to about 2 wt % or about 1.9 wt %. Unit dosage formulations of the embodiments of the present disclosure can include cannabinoid, for example, a phytocannabinoid (including for example, CBD or full spectrum hemp or CBD oil) in the amount of about 2 mg. to about 60 mg., about 5 mg. to about 30 mg., about 5 mg to about 15 mg., about 15 mg. to about 30 mg. or about 30 mg. to about 45 mg. Unit dosage formulations of the embodiments of the present disclosure can include CBD in the amount of about 2 mg. to about 30 mg., about 5 mg. to about 30 mg., about 5 mg. to about 15 mg., about 15 mg. to about 30 mg. or about 10 mg. Unit doses of full spectrum CBD or hemp oil content can include an amount of about 2 mg. to about 60 mg. An effective amount of cannabinoid includes an analgesic, pain relieving, pain treating or anti-inflammatory amount of cannabinoid.

Cannabinoids, for example, CBD can have a local and/or a systemic effect and may reduce pain imparting and regulating the endocannabinoid (neurotransmitter of the nervous system) receptor activity. The subsequent body functions that may be regulated include pain, sleep, appetite and immune system response (through, at least, in part, by reducing inflammation).

For the purpose of the present disclosure, the word “cannabinoid” refers to one or more cannabinoids or cannabinoid compounds or oils or extracts from plants (for example, hemp including hemp oil, full spectrum hemp oil, CBD oil, full spectrum CBD oil, Cannabis sativa seed oil, etc.) that include one or a plurality of phytocannabinoids.

Hemp oil and full spectrum hemp oil is oil derived from the entire hemp plant except the flower (which contains tetrahydrocannabinol (THC)) and can have over 85 phytocannabinoids which can have a positive synergistic effect as compared to compositions having fewer cannabinoids. There may also be benefits to other components of it (e.g., terpenes). Such benefits and effects may include faster penetration and/or permeation of the therapeutic components thereof. Full spectrum hemp oil can include full spectrum hemp oil that has been purified to include less than the below stated amounts of one or more of the following impurities:
Aflatoxins BI, 82, G1, G2 (fats, oils, lecithin, egg powder): <0.1 μg/kg of each of
Aflatoxin B1, Aflatoxin B2, Aflatoxin GI and Aflatoxin G2, Sum of all positive
Aflatoxins <0.4 μg/kg.
GlyphosatelAMPAiGlufosinate: <0.1 mg/kg of each of Glufosinate, Glyphosate and
Aminomethylphosphonic acid (AMPA)
Mercury: <0.02 mg/kg
Arsentic: <0.03 mg/kg
Cadmium: <0.01 mg/kg
Lead: <0.05 mg/kg.

Hemp oil and full spectrum hemp oil can include less than about 0.3 wt % THC.

Polyamines, particularly epsilon-poly-l-lysine (ε-PL) is a natural cationic peptide which is generally regarded as safe (GRAS-generally regarded as safe). It can be used as a natural antimicrobial for foods and personal care products.

In one embodiment of the present disclosure, the natural and synthetic sulfated polymers are crosslinked with cationic polyamines by forming an amine salt with the sulfonated group of the natural and synthetic sulfated polymers and still achieve static yield stress of from about 4.0 Pa. (Pascals) to about 8.0 Pa or about 6.0 Pa. This prevents solution cold creep and keeps particulates suspended at lower polymer concentrations.

In another embodiment of the present disclosure, carrageenan is crosslinked with e-polylysine forming an amine salt with the sulfonated grouped of the carrageenan and will still achieve yield stress of from about 4.0 Pa to about 8.0 Pa or about 6.0 Pa. This prevents the solution from cold creep and keeps particulates suspended at lower polymer concentrations.

The typical gelling concentration of carrageenan is from about 2.0 wt % to about 4.0 wt %. This forms a semi-solid gel with static yield stress properties ranging from about 4.0 Pa to about 8.0 Pa. However, at these concentrations, the skin feel can be tacky and sticky. lota and lambda carrageenan are tackier and slimier. However, by crosslinking carrageenan with a polyamine, such as, for example, e-polylysine, the same static yield stress (properties ranging from about 4.0 Pa) to about 8.0 Pa) can be achieved, but at concentrations of carrageenan less than about 2 wt %. This results in an improvement in skin feel because of a lower concentration of carrageenan. Additionally, the polymer-polymer complex yield stress prevents the agglomeration of exosomes by keeping them apart, even at low polymer-polymer concentrations.

The liposomes used in embodiments of the present disclosure can be in an amount of about 1.00 wt % to about 20.00 wt %, about 5.0 wt % to about 15 wt %, about 10 wt %.

Embodiments of the present disclosure include mixtures where the exosomes and polymer-polymer complex are located inside the liposome, as well as mixtures where the exosomes and polymer-polymer complex are located both inside and outside the liposome.

Embodiments of the present disclosure include crosslinked carrageenan compositions and methods of making a composition that includes mixing together (a) one or more natural or synthetic sulfated polymers, for example, carrageenan (iota or kappa, or lambda) and (b) a crosslinking agent polyamine compound in a ratio of (a) about 1 molar part carrageenan to (b) the amount of one or more than one of a cationic polyamine compound, for example, ε-poly-L-lysine, 0.05 molar part to about 1.00 molar part, about 0.100 molar part to about 0.750 molar part, about 0.150 molar part to about 0.500 molar part, about 0.200 molar part to about 0.300 molar part, or about 0.250 molar part. The anionic sulfated group of the natural and synthetic sulfated polymers, e.g., carrageenan will form an amine salt with the cationic group of the cationic polyamine, e.g., ε-poly-L-lysine to form a crosslinked network that increases the yield stress of the gel from about 4.0 Pa to about 8.0 Pa or about 6.0 Pa. Additionally, monovalent and polyvalent metal ion compounds (for example, lithium hydroxide, sodium hydroxide, potassium hydroxide, magnesium oxide, calcium oxide and zinc oxide) can be used to adjust the pH of the solution to neutral or about pH 7. Metal salts such as sodium hydroxide and/or potassium hydroxide can be added to achieve about pH 7. For example, sodium hydroxide can be added to about 0.05 molar part to about 1.00 molar part, about 0.100 molar part to about 0.750 molar part, about 0.150 molar part to about 0.500 molar part, about 0.200 molar part to about 0.300 molar part, or about 0.250 molar part.

The natural and synthetic sulfated polymer/cationic polyamine composition can then be dried and, afterwards, have the exosome serum added to it.

In one embodiment of the present disclosure, below is an example of the polymer-polymer (e.g., carrageenan-polylysine salt) complexation process:

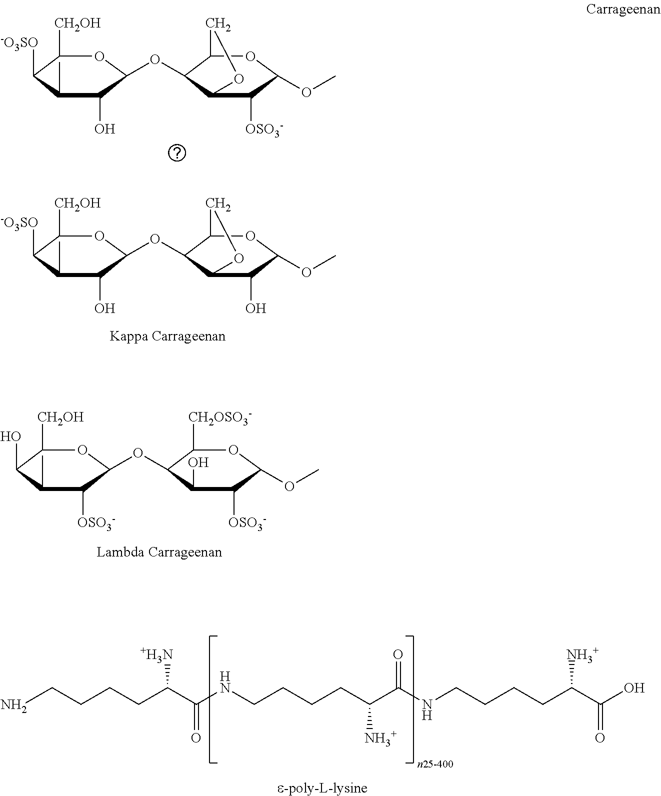

Crosslinked partial salt between carrageenan and ε-poly-L-lysine

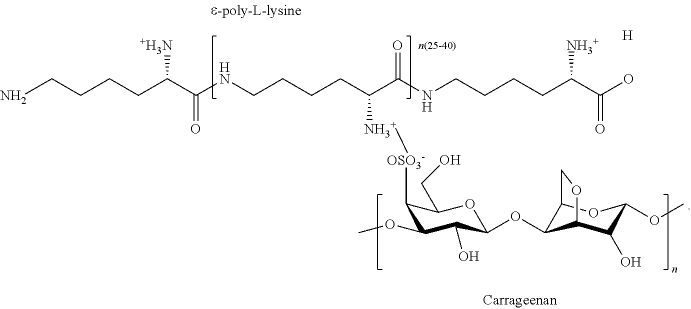

Using the above process, the cationic polyamine, for example, ε-poly-L-lysine, can also connect to another natural or synthetic sulfated polymers, for example, carrageenan, as can the cationic polyamine, for example, ε-poly-L-lysine, can connect to another natural and synthetic sulfated polymers, for example, carrageenan.

In another embodiment of the present disclosure, in a suitable jacketed mixing kettle with stirring/agitation and capable of heating solutions, about 96.7 w/w % of DI water and about 0.09 w/w % sodium hydroxide is added and heated to between about 140° F. and about 160° F., preferably 150° F. A dry premix of about 3.0 w/w % carrageenan and about 0.21 w/w % ε-poly-L-lysine powder is then added to a dry powder mixer such as a drum tumble mixer or equivalent for about 15 to about 20 minutes. The speed of the mixing kettle is set to produce a vortex. To avoid lumps or fisheyes, the carrageenan/ε-poly-L-lysine powder premix is slowly added into the vortex while maintaining a solution temperature of about 150° F. Mixing then takes place for about 30 minutes with strong agitation while maintaining a temperature of about 150° F. until the premix powder is dissolved. The resulting solution is then dried while maintaining a temperature of about 150° F. during the drying process until dry flakes or powders are produced.

The drying equipment that can be used includes, for example, drum dryers, film casting tunnel drying, or spray drying; flash off the water. Moisture content of the flakes or powder should be between about 5 wt % and about 8 wt %. If flakes are produced, milling to a fine powder may be necessary using hammermills, centrifugal mills, or air-impact (jet) mills.

Exosome serum can then be admixed with the carrageenan-polylysine salt as a paste premix which can then be added to topical gel and lotion formulations and other delivery systems such as patches. Transdermal delivery systems, such as topical gels, lotions, patches with or without antiseptics and/or antibiotics such as chlorhexidine, OCT, hypochlorite, PHMB, PVP-I, HOCl, bacitracin, neomycin, and polymixin b, benzalkonium chloride, etc. can be used for the treating or prevention of infections of minor cuts, scrapes, wounds, and burns, and promoting wound and burn healing.

Transdermal delivery systems, patches, vehicles and devices as well as methods of use and methods of treatment using same and methods of manufacture of same, such as, for example, embodiments of the present disclosure, include transdermal delivery systems, patches, vehicles and devices can be placed topically on a body part (e.g., arm, leg, knee, torso, head, neck, foot as well as those parts that make-up them) for purposes of local and/or systemic administration for a time sufficient to be effective for purposes of therapeutic activity to the body part and tissues thereof or other tissues remote from the application site in order to provide relief from the malady being suffered including a malady of the body part (e.g., treating or prevention of infections in minor cuts, scrapes, wounds, and burns, and promoting wound and burn healing) to which the transdermal delivery systems, patches, vehicles and devices of the present disclosure can be directly applied for relief. After being present in contact with the body part for a time sufficient to be effective for purposes of therapeutic activity, they can be removed from the body part. Such application to the body part includes placing the transdermal delivery systems, patches, vehicles and devices in contact with the skin covering the body part.

Embodiments of the present disclosure may be delivered for local or systemic administration to a body part of a person to be treated with the embodiment, for example, a body or skin surface thereof by placing an embodiment of the present disclosure on a body part or skin surface thereof, for example, a knee, leg, back of hand, arm, lower back,, upper back, shoulder and forehead, for the treating or prevention of infections in minor cuts, scrapes, wounds, and burns, and promoting wound and burn healing.

Embodiments of the present disclosure also include methods of use of the exosome composition embodiments of the present disclosure that are applied to bone and tissue and used to promote wound healing as well as bone and tissue growth, for example, used in surgical tissue and bone graft procedures. Embodiments of the present disclosure can also be topically applied to the skin of a person, for example, to the scalp or other skin surface, to promote hair growth as well as to treat skin defects such as aging, atopic dermatitis, and wounds.

Embodiments of the present disclosure can also include additional therapeutic compositions for topical or internal administration that may also include other therapeutic or pharmaceutical actives such as, for example, menthol, camphor and cannabinoids (e.g., hemp oil, cannabidiol) as well as the methods of making and using/treating using such therapeutic compositions. The majority of beneficial components are in the exosomes. However, stem cells also secrete cytokines and growth factors (natural components) into the surrounding medium which are not enclosed in exosomes. If liposomes are created with exosomes and growth factors/cytokines present, then these can also be enclosed into the liposomes.

Methods of use of embodiments of the present disclosure can include gastrointestinal delivery (oral delivery, sublingual delivery, buccal delivery, rectal delivery), parenteral delivery (intradermal delivery, sub-cutaneous delivery, intramuscular delivery, intravenous delivery) and Topical (transdermal patches, instillations, irrigation or douching, epidermic or enepidermic routes, throat paints, inhilation routes).

The embodiments of the present disclosure including exosomes can be made at about room temperature by combining the above described exosome suspensions in approximately isotonic aqueous solutions of, for example, 0.9 wt % sodium or phosphate buffered saline with a solvent/skin penetrant (e.g., DMSO) and the polymer-polymer complex of the present disclosure and optionally additional ingredients disclosed herein (e.g., humectant/emollients, thickeners, chelating agents, etc.). Additional water at about room temperature can be added (isotonic or distilled) to adjust the exosome concentration. Buffers can be added at about room temperature to adjust the pH, if necessary, to between about 5.0 to about 7.5, which is a critical range in which the exosomes should reside. The pH can be adjusted to be within this range using buffers such as ammonium sulfate, sodium citrate, sodium chloride, sodium acetate.

The salinity can then optionally be adjusted at about room temperature to make the resulting mixture approximately isotonic. The resulting mixture can then be maintained at between about −200° C. and room temperature, preferably, frozen if to be stored.

The embodiments of the present disclosure including exosomes and liposomes and/or nicosomes where it is desired to have liposomes and/or nicosomes with exosomes included therein can be made at about room temperature by combining the above described exosome suspensions in approximately isotonic aqueous solutions of, for example, 0.9 wt % sodium or phosphate buffered saline with a polymer-polymer complex of the present disclosure. The polymer-polymer complex of the present disclosure is formed to form a first mixture. The exosomes are then added to the first mixture with the polymer-polymer complex of the present disclosure to form a second mixture. The second mixture is added to at least one lipid to form the liposomes and/or nicosomes (e.g., lecithin alone or in combination with zeolite). Then a solvent/skin penetrant (e.g., DMSO) and preservative (e.g., epsilon poly L-lysine) and optionally additional ingredients disclosed herein (e.g., humectant/emollients, thickeners, chelating agents, etc.) are added at about room temperature. If it is desired to have exosomes also external to the liposomes and/or nicosomes, second mixture of exosomes and the polymer-polymer complex of the present disclosure can also be added after the liposomes and/or nicosomes are formed. Additional water can be added at about room temperature (isotonic or distilled) to adjust the exosome concentration. Buffers can be added at about room temperature to adjust the pH, if necessary, to between about 5.0 to about 7.5. The salinity can then optionally be adjusted at about room temperature to make the resulting mixture approximately isotonic. The resulting mixture can then be maintained at between about −200° C. and room temperature, preferably, frozen if to be stored.

The terms “treating” and “effective amount”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. In a further embodiment, a kit is disclosed. One example of such a kit is a kit including one or more of the embodiments of the present disclosure separately packaged and instructions for use.

Another embodiment of the present disclosure includes the treatment or prevention of infections in minor cuts, scrapes, wounds, and burns, and promoting wound and burn healing. This method includes applying one of the exosome containing embodiments of the present disclosure to a cut, scrape, wound, abrasion, burn or other external wound of an animal (e.g., a human or other animal).

### EXAMPLES

TABLE 1

| | Exosome Gel Composition | | | | |
|---|---|---|---|---|---|
| Item# | Ingredients | Function | w/w % | lower | upper |
| 1 | DI Water | Solvent | 93.79 | 90.00 | 95.00 |
| 2 | PEG-12 dimethicone | Surfactant | 2.00 | 1.00 | 3.00 |
| 3 | Butylene glycol | Humectant | 1.30 | 1.00 | 5.00 |
| 4 | Carrageenan/Polylysine | Polysaccharide | 1.06 | 0.50 | 1.50 |
| 5 | Exosome** | Active | 1.00 | 0.10 | 1.50 |
| 6 | DMSO | Solvent | 0.50 | 0.05 | 1.00 |
| 7 | Linatural | Preservative | 0.20 | 0.10 | 0.30 |
| 8 | Benzalkonium chloride | Antibacterial/ | 0.12 | 0.10 | 1.00 |
| | | Antiseptic | | | |
| 9 | Sodium hydroxide or | pH adjuster | 0.03 | 0.01 | 0.10 |
| | Citric acid | | | | |
| | | TOTAL | 100.00 | | |

TABLE 2

| | Exosome Lotion Composition | | | | |
|---|---|---|---|---|---|
| Item# | Ingredients | Function | w/w % | upper | lower |
| 1 | Avocado oil | Emollient | 46.00 | 40.00 | 50.00 |
| 2 | DI Water | Solvent | 25.85 | 20.00 | 30.00 |
| 3 | Beeswax | Emulsifier | 15.00 | 10.00 | 20.00 |
| 4 | Aloe Vera Extract | Humectant | 5.24 | 1.00 | 10.00 |
| 5 | PEG-12 dimethicone | Surfactant | 3.00 | 1.00 | 5.00 |
| 6 | Coconut oil | Emollient | 2.00 | 1.00 | 3.00 |
| 7 | Carrageenan/Polylysine | Polysaccharide | 1.06 | 0.50 | 1.50 |
| 8 | Exosome** | Active | 1.00 | 0.50 | 1.50 |
| 9 | DMSO | Solvent | 0.50 | 0.10 | 1.00 |
| 10 | Linatural | Preservative | 0.20 | 0.10 | 0.30 |
| 11 | Benzalkonium chloride | Antibacterial/ | 0.12 | 0.10 | 1.00 |
| | | Antiseptic | | | |
| 12 | Sodium hydroxide or | pH adjuster | 0.03 | 0.01 | 0.10 |
| | Citric acid | | | | |
| | | TOTAL | 100.00 | | |

TABLE 3

| | Exosome Silicone-based Lotion Composition | | | | |
|---|---|---|---|---|---|
| Item# | Ingredients | Function | w/w % | upper | lower |
| 1 | Dimethicone | Emollient | 91.58 | 85.00 | 95.00 |
| 2 | Aloe Vera Extract | Humectant | 0.50 | 0.10 | 1.00 |
| 3 | PEG-12 dimethicone | Surfactant | 5.00 | 2.50 | 7.50 |
| 4 | Carrageenan/Polylysine | Polysaccharide | 1.10 | 0.50 | 1.50 |
| 5 | Exosome** | Active | 1.00 | 0.50 | 1.50 |
| 6 | DMSO | Solvent | 0.50 | 0.10 | 1.00 |
| 7 | Linatural | Preservative | 0.20 | 0.10 | 0.30 |
| 8 | Benzalkonium chloride | Antibacterial/ | 0.12 | 0.10 | 1.00 |
| | | Antiseptic | | | |
| | | TOTAL | 100.00 | | |

TABLE 4

| | Peelable Silicone Film Exosome Composition | | | | |
|---|---|---|---|---|---|
| Item# | Ingredients | Function | w/w % | upper | lower |
| 1 | Acrylates dimethicone | Elastomer | 50.00 | 45.00 | 55.00 |
| 2 | Cyclomethicone | Diluent | 39.28 | 35.00 | 45.00 |
| 3 | Fumed Silica | Filler | 5.00 | 2.50 | 7.50 |
| 4 | PEG-12 dimethicone | Surfactant | 3.00 | 2.00 | 4.00 |
| 5 | Carrageenan/Polylysine | Polysaccharide | 1.10 | 0.50 | 1.50 |
| 6 | Exosome** | Active | 1.00 | 0.50 | 1.50 |
| 7 | DMSO | Solvent | 0.50 | 0.10 | 1.00 |
| 8 | Benzalkonium chloride | Antibacterial/ | 0.12 | 0.10 | 1.00 |
| | | Antiseptic | | | |
| | | TOTAL | 100.00 | | |

TABLE 5

| | Peelable Exosome Film Composition | | | | |
|---|---|---|---|---|---|
| Item# | Ingredients | Function | w/w % | upper | lower |
| 1 | DI Water | Solvent | 79.09 | 75.00 | 85.00 |
| 2 | Polyvinyl alcohol | Elastomer | 12.00 | 10.00 | 20.00 |
| 3 | PEG-12 dimethicone | Surfactant | 3.00 | 2.00 | 4.00 |
| 4 | Butylene glycol | Humectant | 3.00 | 2.50 | 3.50 |
| 5 | Carrageenan/Polylysine | Polysaccharide | 1.06 | 0.50 | 1.50 |
| 6 | Exosome** | Active | 1.00 | 0.50 | 1.50 |
| 7 | DMSO | Solvent | 0.50 | 0.10 | 1.00 |
| 8 | Linatural | Preservative | 0.20 | 0.10 | 0.30 |
| 9 | Benzalkonium chloride | Antibacterial/ | 0.12 | 0.10 | 1.00 |
| | | Antiseptic | | | |
| 10 | Sodium hydroxide or | pH adjuster | 0.03 | 0.01 | 0.10 |
| | Citric acid | | | | |
| | | TOTAL | 100.00 | | |

Thus, while there have been shown, described and pointed out, fundamental novel features of the present disclosure as applied to the exemplary embodiments thereof, it will be understood that various omissions and substitutions and changes in the form and details of devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

This written description uses examples as part of the disclosure, including the best mode, and also to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

While there have been shown, described and pointed out, fundamental features of the present disclosure as applied to the exemplary embodiments thereof, it will be understood that various omissions and substitutions and changes in the form and details of compositions, devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

1. A composition, comprising:
a plurality of exosomes; and
a polymer-polymer complex including one or more natural or synthetic sulfated polymers crosslinked with one or more crosslinking agent cationic polyamines.

2. The composition of claim 1, wherein the natural or synthetic sulfated polymer includes carrageenan.

3. The composition of claim 1, wherein the cationic polyamine includes ¿-poly-L-lysine.

4. The composition of claim 1, wherein the crosslinking agent polyamine compound is in a ratio of (a) about 1 molar part the one or more natural or synthetic sulfated polymers to (b) 0.05 molar part to about 1.00 molar part crosslinking agent polyamine compound.

5. The composition of claim 1, wherein the plurality of exosomes includes placental cell exosomes, stem cell exosomes or a mixture thereof.

6. The composition of claim 1, wherein the plurality of exosomes includes plant exosomes.

7. The composition of claim 1, wherein the plurality of exosomes is present in an amount of about 0.25 wt % to about 1.00 wt %, about 0.50 wt %, about 10 million to about 20 trillion exosomes per 1 ml volume or about 1 billion exosomes per 1 ml volume.

8. The composition of claim 1, wherein the plurality of exosomes and the polymer-polymer complex are positioned inside a plurality of liposomes or both outside and inside the plurality of liposomes.

9. A method, comprising:
using a composition including
a plurality of exosomes; and
a polymer-polymer complex including one or more natural and synthetic sulfated polymers crosslinked with one or more crosslinking agent cationic polyamines; and
applying the composition to a cut, scrape, wound, abrasion, burn or other external wound of an animal.

10. The method of claim 9, wherein the plurality of exosomes includes placental cell exosomes, stem cell exosomes or a mixture thereof.

11. The method of claim 9, wherein the plurality of exosomes includes plant exosomes.

12. The method of claim 9, wherein the natural or synthetic sulfated polymer includes carrageenan.

13. The method of claim 9, wherein the cationic polyamine includes E-poly-L-lysine.

14. The method of claim 9, wherein the crosslinking agent polyamine compound is in a ratio of (a) about 1 molar part the one or more natural or synthetic sulfated polymers to (b) 0.05 molar part to about 1.00 molar part crosslinking agent polyamine compound.

15. A method of making an exosome composition that maintains the structure and integrity of the exosomes, comprising:
mixing
a plurality of exosomes;
a polymer-polymer complex including one or more natural or synthetic sulfated polymers crosslinked with one or more crosslinking agent cationic polyamines to the first mixture to form a second mixture; and
a lipid that forms liposomes without the use of alcohol to the second mixture.

16. The method of claim 15, wherein the plurality of exosomes and the polymer-polymer complex are mixed to form a first mixture and the lipid that forms liposomes without the use of alcohol is added to the first mixture and mixed to form a second mixture.

17. The method of claim 15, wherein the natural and synthetic sulfated polymer includes carrageenan and the cationic polyamine includes E-poly-L-lysine.

18. The method of claim 15, wherein the crosslinking agent polyamine compound is in a ratio of (a) about 1 molar part the one or more natural or synthetic sulfated polymers to (b) 0.05 molar part to about 1.00 molar part crosslinking agent polyamine compound.

19. The method of claim 15, wherein the plurality of exosomes includes placental cell exosomes, stem cell exosomes or a mixture thereof.

20. The method of claim 15, wherein the plurality of exosomes includes plant exosomes.
